(19) United States
(12) Patent Application Publication
(10) Pub. No.: US 2004/0151716 A1
(43) Pub. Date: Aug. 5, 2004

(54) Material and method for treating microbial mediated dermatological conditions

(76) Inventors: Richard A. Hamer, Belleville, MI (US); Craig G. Burkhart, Toledo, OH (US); Craig N. Burkhart, Providence, RI (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/770,091

(22) Filed: Feb. 2, 2004

Related U.S. Application Data
(60) Provisional application No. 60/444,458, filed on Feb. 3, 2003.

(51) Int. Cl. A61K 38/46; A61K 38/47; A61K 38/48; A61N 1/00

(52) U.S. Cl. 424/94.63; 424/94.6; 424/94.61; 514/12; 607/1

(57) ABSTRACT

Microbe mediated dermatological conditions in an organism are treated by the use of an agent which is capable of disrupting a microbial biofilm. The agent may be a chemical agent or a physical agent such as sonic energy or an electrical field. In addition, the tissue may be treated with antimicrobial compound. Also disclosed are therapeutic materials used in the method.

### RELATED APPLICATION

This application claims priority of U.S. Provisional Patent Application Serial No. 60/444,458 filed Feb. 3, 2003, entitled “Material and Method for Treating Microbial Mediated Dermatological Conditions.”

### FIELD OF THE INVENTION

This invention relates generally to the treatment of disease conditions. More specifically, the invention relates to the treatment of dermatological conditions in which bacteria, fungi and other microbes play a role. Most specifically, the invention relates to a method for treating dermatological conditions by control of microbial biofilms.

### BACKGROUND OF THE INVENTION

Microbes have been shown to cause or exacerbate various dermatological conditions. In the context of this disclosure, dermatological conditions are defined to include infections, irritations and other, pathologies of the skin, scalp and nails. Dermatological conditions include, by way of illustration and not limitation, acne, acne rosacea (also referred to generally as rosacea), fungal infections such as dermatophytoma, other onychomycoses, and the like. Various materials and methodologies have been employed for the treatment of such microbial mediated dermatological conditions; however, in many instances, it has been found that topical and/or systemic treatment with antibiotic compositions is only marginally effective in controlling such conditions. In many instances, agents which readily control microbial growth in vitro have been found to have very low efficacy in controlling actual disease conditions caused by these organisms.

The present invention recognizes that biofilms play a heretofore unappreciated role in the development, progress and maintenance of microbe mediated dermatological disease conditions. It has only recently come to be appreciated that microbes often exist and propagate within a biofilm environment. As is understood in the art, biofilms are comprised of bacteria, fungi and other microbes, together with their secretions which can include mucoid-like mixtures of polysaccharides and other microbial metabolites. The biofilm environment shields the microbes from the ambient environment and also provides a conductive medium whereby microbes can establish intercellular chemical communication. This communication is often referred to as quorum sensing, and enables a microbial colony to act in a collective sense to alter its metabolism and other biochemical processes in response to environmental conditions. In addition, the biofilm matrix, which can grow to macroscopic thicknesses, shields the microbes from the action of antimicrobials and like therapeutic agents. Furthermore, it has been found that, in many instances, growth and metabolic rates of microbes in a biofilm can be significantly different from those of free-living organisms; and hence, the vulnerability to therapeutic agents of such cells is further diminished. In addition, biofilms can provide a series of distinct microenvironments in which microbes can express different metabolic rates and biochemical processes; hence, biofilms can allow for the natural selection of diverse microbial strains and variants.

While the role of biofilms in sustaining microbial infections of dental equipment, hydraulic systems, food handling equipment and the like has been recognized in the art, the role of biofilms in sustaining and propagating dermatological pathologies has not been heretofore appreciated.

### BRIEF DESCRIPTION OF THE INVENTION

There is disclosed herein a method for treating a microbe mediated dermatological condition through the use of an agent which is capable of disrupting a microbial biofilm on the affected tissue. In particular instances, the agent is a chemical compound such as guaifenesin, cholate, deoxycholate, aldolase, pepsin, chymostrypsin, trypsin, carboxypeptidase, lipases, amylase, β-galactosidase, lactase, α-glucosidase, sucrase, colipase, pancreatic protein, DNAase, acetylcysteine, peroxide radicals, and combinations thereof. In other instances, the therapeutic agent may comprise a physical agent such as sonic waves or an electrical field.

In addition to the use of the biofilm disrupting agent, the tissue may be treated with an antimicrobial compound either during or after treatment with the microfilm disrupting agent. The method has particular use for the treatment of dermatological conditions such as acne, acne rosacea, and fungal infections of skin and nails. Also disclosed are materials for use in the therapies.

### DETAILED DESCRIPTION OF THE INVENTION

In accord with the present invention, it has been found that microbe mediated dermatological conditions can be successfully treated by the use of agents which remove or disrupt biofilms which may be present on dermal tissue such as skin, scalp and nails. Within the content of this disclosure, disruption of a biofilm is understood to mean any process which compromises the integrity of the biofilm. Disruption need not comprise completely removing a biofilm from a surface, but may comprise breaking the film into separated portions, ablating portions of the thickness of the film, loosening the film from the substrate and the like.

In some instances, the simple disruption of a biofilm will be sufficient to alleviate a disease condition by altering the biofilm environment which supports excessive or undesirable microbes. In other instances, the therapy may be further advanced by the use of antimicrobial materials or any other means.

It has been found that disruption of a dermatological biofilm destroys the microenvironments which support and foster microbial growth thereby making the microbe more vulnerable to antimicrobial therapies or affecting their normal activities. In addition, disruption of the biofilm will affect the metabolic rate of the microbes further enhancing their vulnerability to therapeutic agents. As mentioned above, in many instances, simple disruption of the biofilm will suffice to alleviate or control these conditions. The disruption of the biofilm will permit natural host responses such as immune responses or the generation of biogenic control species such as free radicals, to alleviate the microbial infection. In addition, disruption of the biofilm will expose the microbes to the ambient environment in which they may be vulnerable to the effects of drying, ultraviolet radiation and the like.

There are a variety of agents which may be employed to disrupt a biofilm. As noted above, disruption of a biofilm refers to complete removal of the biofilm, as well as to treatments in which the integrity of the biofilm is compromised. Among the agents which may employed are chemical agents such as surfactant compounds, enzymes, denaturing agents such as halogens or alkylating agents, and the like. Among some of the chemical agents which may be employed are guaifenesin, cholate, deoxycholate, aldolase, pepsin, chymotrypsin, trypsin, carboxypeptidase, lipases, amylase, β-galactosidase, lactase, α-glucosidase, sucrase, colipase, pancreatic proteins, DNAase, acetylcysteine, peroxide radicals and the like. These agents may be employed either singly or in combinations. One particularly preferred material comprises guaifenesin. This compound is an effective mucolytic, and its safety and efficacy have been well established. This material has been widely used as an expectorant for the treatment of a variety of respiratory conditions; however, guaifenesin has not heretofore been incorporated into dermatological compositions, and more specifically has never been used to treat acne, fungal infections, and other such dermatological conditions in which microbial infection plays a role.

Physical agents such as ultrasonic energy can be employed to disrupt biofilms, and such ultrasonic energy can be readily applied to skin and nails by an ultrasonic transducer appropriately coupled thereto by a thin film of fluid, which fluid may further contain a therapeutic agent such as a chemical disrupting agent and/or an antimicrobial. Biofilms are comprised of complex macromolecules, which are generally responsive to an electrical field, and, in accordance with the present invention, biofilms may also be disrupted by an applied field. For example, a series of laterally spaced electrodes may be applied to the surface of the skin and the field established therebetween. This field will disrupt the biofilm. The field is preferably coupled into the biofilm by a fluid electrolyte which may contain other therapeutic agents. Alternatively, the field may be imposed in a direction perpendicular to the dermal surface, and such a field may be employed to transport therapeutic materials, such as biofilm disrupting compounds as well as antimicrobials into and through the biofilm.

There are a number of microbes associated with dermatological disease conditions which may be treated by the present invention; and accordingly, the choice of antimicrobial compound employed for use in the present invention will depend upon the specific microbe or microbes which are involved. In general, the antimicrobial compound may comprise an antibiotic material such as clindamycin, erythromycin, penicillin or any other such antibiotic. In those instances where the microbe involved is a fungus, antifungal material such as terbinafine or the like may be utilized as the antimicrobial. Antiseptic materials such as phenols, halogens, peroxides and other disinfectant materials may also be employed as an antimicrobial. In some instances, a combination of antimicrobial materials may be utilized in the practice of the present invention.

Various treatment regimens may be implemented in accord with the present invention. In some instances, a unitary treatment comprising a combination of a biofilm disrupting agent and an antimicrobial agent may be employed. As discussed above, the biofilm disrupting agent may comprise one or more agents, as may the antimicrobial. In other instances, sequential treatment regimens may be preferred. In such instances, the biofilm disrupting agent is employed in a first stage of the treatment, and thereafter antimicrobials are employed. Various hybrid combinations of these two regimens will be readily apparent to one of skill in the art, and are within the scope of this invention. Treatment possibilities are not limited to what is specifically disclosed herein. Other treatments and methods will be apparent to one of skill in the art in view of the disclosure and teaching of this provisional patent application.

The therapeutic materials and methods of the present invention may be employed with particular advantage for the treatment of acne. Heretofore, acne treatments were primarily directed to eradicating Propionibacterium acnes from the anaerobic environment of the hair units in which they reside. Such efforts have been notoriously ineffective. It has now been determined that the P acnes bacteria is part of normal skin flora, and its presence, in appropriate amounts, is desirable. In view of this paradigm, it will now be understood why agents which are highly effective in controlling P acnes in vitro can be ineffective in vivo. It will also be appreciated that disruption of the biofilm will allow for the control of P acnes so as to restore the normal balance of skin flora.

One highly effective group of agents for the disruption of biofilms associated with dermatological conditions comprises peroxide radicals, and one preferred group of peroxide radicals comprises organic peroxide radicals such as benzoyl peroxide radicals. It has been found that such radicals can be generated from benzoyl peroxide by treatment with active amine compounds. One particularly effective amine comprises the antifungal drug naftifine, and another comprises terbinafine. Specifically, topical compositions comprising combinations of benzoyl peroxide and naftifine or terbinafine, in an appropriate vehicle, have been found to generate peroxide radicals which disrupt the biofilm permitting access of the topically applied terbinafine to the infected sites. In addition, the benzoyl peroxide, and radicals thereof, also exert an antimicrobial effect. Other amine compounds, including tertiary amines and allylamines, will also be effective in generating peroxide radicals; and it is anticipated that still other compounds having active nitrogen sites may be employed to generate the peroxide radicals.

Another group of dermal conditions which are notoriously difficult to control are fungal infections, particularly fungal infections of the nails. Such infections can become very persistent, and are resistant to topical treatments. In many instances, oral antifungals such as terbinafine must be employed to treat nail infections; and even in such instances, long-term treatment with oral medications is required. In other instances, fungal infections of the nails can only be treated by surgically removing the nail bed and applying topical agents directly thereto. In accord with the present invention, it has been found that topical treatment of dermatophytoma and other such onychomycoses may be accomplished through the present invention. In many instances, dermatological infections, such as nail fungal infections, are further complicated by the fact that the infected site may be colonized by both bacteria and fungi, and the methodology of the present invention will effectively address both infections.

The present invention may also be employed for the treatment of other dermatological conditions such as dandruff, acne rosacea (also known as rosacea), athlete's foot and the like. It will be appreciated that the present invention represents a new paradigm in the treatment of dermatological infections insofar as it recognizes that biofilms play a very important role in the sustenance and propagation of microbial infections; and that disruption of the biofilm is a very important part of any treatment protocol. While this disclosure is directed to some specific conditions, treatment methodologies and materials, it is to be understood that in view of the teaching herein, yet other modifications and variations of the invention will be readily apparent to one of skill in the art. The foregoing is illustrative of the invention, but is not meant to be a limitation upon the practice thereof. It is the following claims, including all equivalents, which define the scope of the invention.

1. A method for treating a microbe mediated dermatological condition in an organism, said method comprising:
treating an affected tissue of said organism with an agent which is capable of disrupting a microbial biofilm.

2. The method of claim 1, wherein said agent is selected from the group consisting of: guaifenesin, cholate, deoxycholate, aldolase, pepsin, chymotrypsin, trypsin, carboxypeptidase, lipases, amylase, β-galactosidase, lactase, α-glucosidase, sucrase, colipase, pancreatic protein, DNAase, acetylcysteine, peroxide radicals, and combinations thereof.

3. The method of claim 1, wherein said agent comprises sonic waves.

4. The method of claim 1, wherein said agent comprises an electrical field.

5. The method of claim 1, including the further step of treating said affected tissue with an antimicrobial compound.

6. The method of claim 5, wherein said step of treating said affected tissue with an antimicrobial compound comprises treating said tissue with said antimicrobial compound at the same time that the tissue is treated with said agent.

7. The method of claim 5, wherein said step of treating said tissue with an antimicrobial compound is implemented after said tissue is treated with said agent.

8. The method of claim 1, wherein said dermatological condition is selected from the group consisting of: acne, acne rosacea, and fungal infections.

9. The method of claim 1, wherein said affected tissue comprises skin.

10. The method of claim 1, wherein said affected tissue comprises nail tissue, and said dermatological condition comprises dermatophytoma.

11. A therapeutic material for treating a microbe mediated dermatological condition in a tissue of an organism, said therapeutic material comprising:
a topical agent selected from the group consisting of: guaifenesin, cholate, deoxycholate, aldolase, pepsin, chymotrypsin, trypsin, carboxypeptidase, lipases, amylase, β-galactosidase, lactase, α-glucosidase, sucrase, colipase, pancreatic protein, DNAase, acetylcysteine, peroxide radicals, and combinations thereof, said agent being present in an amount sufficient to disrupt any biofilm present on the surface of said tissue; and
an antimicrobial compound.

12. The therapeutic material of claim 11, wherein said antimicrobial compound is selected from the group consisting of antibiotics, antifungals, antiseptics, fungistatic agents, bacteriostatic agents, and combinations thereof.

13. The therapeutic material of claim 11, wherein said agent comprises guaifenesin.

14. The therapeutic material of claim 11, wherein said antimicrobial compound comprises terbinafine.

15. The therapeutic material of claim 11, wherein said agent comprises peroxide radicals.

16. The therapeutic material of claim 15, wherein said peroxide radicals comprise organic peroxide radicals.

17. The therapeutic material of claim 16, wherein said organic peroxide radicals comprise benzoyl peroxide radicals.

18. The therapeutic material of claim 17, wherein said benzoyl peroxide radicals are produced by the interaction of benzoyl peroxide with an amine.

19. The therapeutic material of claim 18, wherein said amine is terbinafine.
